(19) United States
(12) Patent Application Publication
(10) Pub. No.: US 2003/0126651 A1
(43) Pub. Date: Jul. 3, 2003

(54) Chrysanthemum variety 95-105-6

(76) Inventors: Neil Owen Anderson, St. Paul, MN (US); Peter David Ascher, St. Paul, MN (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 09/504,337

(22) Filed: Feb. 14, 2000

(51) Int. Cl. A01H 5/00

(52) U.S. Cl. 800/323

(57) ABSTRACT

The present invention relates to new, distinct and stable cultivar of chrysanthemum.

### FIELD OF THE INVENTION

The present invention relates to a new and distinct cultivar of chrysanthemum, Dendranthema x hybrida, which is referred to as 95-105-6. Chrysanthemum variety 95-105-6 exhibits a shrub-like appearance with a massive floral display during its second year of growth. Additionally, 95-105-6 exhibits superior winter hardiness and frost tolerance in zones 3-10 and is self-pinching.

The present invention also relates to seed, pollen, cuttings and ovules of the chrysanthemum variety 95-105-6. Moreover, the present invention also relates to a tissue culture comprising regenerable cells of chrysanthemum variety 95-105-6.

The present invention also relates to methods for producing F₁ hybrid chrysanthemum seed using chrysanthemum plant 95-105-6 in breeding as either a female or male parent. The present invention also relates to a F₁ hybrid or later generation chrysanthemum plant grown from the hybrid chrysanthemum seed produced by the aforementioned methods.

### BACKGROUND OF THE INVENTION

Cultivated garden and greenhouse chrysanthemums are generally classified in the genus Dendranthema grandiflora Tzvelv. (Chrysanthemum morifolium Ramat.) in the Asteraceae family. In some instances, breeders have crossed Dendranthema grandiflora with other members of the Anthemideae tribe, to which D. grandiflora belongs, to introduce desirable traits.

The actual origin of the garden mum is uncertain. Cultivated chrysanthemums are complex allopolyploids (2n=6x=54) with somatic chromosome numbers ranging from 2n=47-63 due to aneuploidy. Interspecific fertility, morphological, and ecological evidence let Shimotomai to suggest that chrysanthemums originated from two or three diploid ancestors (Shimotomai, N. Journal of Science, Hiroshima University, Series B, Division 2, 1:1-100 (1933)). However, other researchers believe that Dendranthema grandiflora is descended from the two hexaploid Chinese species, Chrysanthemum indicum and Chrysanthemum morifolium Ramat. (C. sinense Sabine), through repeated cycles of hybridization and selection over more than two millenia (Dowrick, G. J., Heredity 6:365-375 (1952); Nazeer, M. A. and Khoshoo, T. N., Current Science, 51:583-85 (1982)). Ackerson (Ackerson, C., National Chrysanthemum Society 23(3):105-107 (1967)) claims that the ancestry of Dendranthema grandiflora includes at least ten species.

Records of chrysanthemum culture in China for pharmaceutical purposes date to more than 5000 years ago. In 385 AD, Korea bestowed a gift of chrysanthemum seeds on Japan (Ackerson, C., National Chrysanthemum Society, 23(3): 105-107 (1967)). The Japanese adopted chrysanthemum culture as a pastime, and are responsible for the development of many of the unique flower forms now observed. Chrysanthemums were introduced to Europe in 1688 by the Dutch. During the 1700's the practice of cultivating chrysanthemums spread from the Netherlands to Great Britain and the United States (Clark, R. B., National Chrysanthemum Society, 18(3):144(1962)).

In 1827, following several plant collection trips earlier in the century to China, Europeans began hybridization and propagation of chrysanthemums from seed (Woolman, J., Chrysanthemum for Garden and Exhibition. Collinridge Ltd., London (1953)). Japanese chrysanthemums were introduced into Europe in 1861 (Fogg, H. G. W., Chrysanthemum Growing, Butler and Tanner Ltd., London (1962)). The Japanese accessions were hybridized with the plants of Chinese origin, giving rise to the European-style chrysanthemums.

Following the work of Garner and Allard (Garner, W. W. and Allard, H. A., Flowering and Fruiting of Plants Controlled by the Day, 852 Yearbook, U.S. Dept. of Agriculture (1920)) on photoperiodic responses implants, protocols were developed for forcing chrysanthemums flowering throughout the year (Duffett, W. E., The Breeder's Handbook, National Chrysanthemum Society (1957)). As a consequence, the culture and breeding of chrysanthemums has been split into greenhouse and garden mum production. Greenhouse mums are further divided into florist pot mums and cut flowers. Florist pot mums and cut flowers are sold year round with emphasis on holiday sales at Christmas and Mother's Day. Cut mums are popular flowers for floral designers because they are relatively inexpensive and have long vase life. Garden mum sales are strongest from mid-summer through the fall. They have always been popular as a means of adding color to gardens in the fall when little else is in bloom. Mums are widely used both by the home gardener as well as in public gardens. There has recently been a greater use of chrysanthemums in hanging baskets, planter boxes, and patio containers (Higgins, E., Tips on Growing and Marketing Garden Mums, Ohio Florists' Association (1996)).

There are currently several thousand cultivars of garden and greenhouse chrysanthemums available (National Chrysanthemum Society, Handbook on Chrysanthemum Classification (1996)). The quantity of garden mums sold wholesale in 1997 was 46.9 million, compared with sales of pot mums of 27.6 million, and of cuts of 12.1 million blooms. The value of garden mums sold was $73.2 million, of pot mums was $82.8 million, and of cuts mums was $5.8 million. Production of garden mum has increased by 17% from 1993-1997, while production of pot mums and cut mums have decreased by 16% and 63% respectively. The wholesale value of garden mums has increased at a faster rate than total production has increased, due to the tendency of growers to market mums in larger container sizes for which consumers are willing to pay higher prices. The total dollar value of pot mums sold is greater that of garden mums because pot mums are sold for prices two to three times that charged for garden mums in the same size pots (National Agricultural Statistics Service. 1998. Floriculture Crops Summary, USDA)).

Chrysanthemums have a wide array of flower forms. The chrysanthemum inflorescence is composed of a central disk made up of a few to several hundred hermaphroditic florets surrounded by one or more rows of ray florets that are lacking anthers. The National Chrysanthemum Society (National Chrysanthemum Society, Handbook on Chrysanthemum Classification (1996)) classifies chrysanthemum inflorescence into thirteen classes. Classes are distinguished by the size of the flowers, the shape of the ray petals, the degree of doubleness of the inflorescence, i.e. the degree to which the disk is visible, and the way in which the petals curve. Classes listed are irregular incurve, reflex, regular incurve, decorative, intermediate, pompon, single or semi-double, anemone, spoon, quill, spider, brush or thistle, and unclassified (not fitting any other class). Greenhouse chrysanthemums typically have a broader range of flower forms than garden mums, although breeding efforts have been and are being made to incorporate the greenhouse forms into garden chrysanthemums (Viehmeyer, G., The Breeder's Handbook, National Chrysanthemum Society (1957); Glicenstein, L., Tips on Growing and Marketing Garden Mums, Ohio Florists' Association (1996)).

Chrysanthemums come in five basic color classes: white, purple, red, bronze and yellow. Typical plant habits include cushions, mounds and upright forms. Blooming dates for mums when grown outside under natural daylengths range from 5-12 weeks from floral initiation to full flowers.

In addition to flower forms, chrysanthemum breeders are also focusing on earliness of flowering under natural daylengths, development of varieties that bloom after the conventional mum season has passed (in winter climates), winter hardiness, and disease and insect resistance. The current interest by consumers for novel plant habits has encouraged breeders to work on developing new plant forms for chrysanthemums. Breeders have used interspecific crosses to develop new plant habits and to improve winter hardiness and frost intolerance (Glicenstein, L., Tips on Growing and Marketing Garden Mums, Ohio Florists' Association (1996); Ascher, P. and Strefeler, M., Tips on Growing and Marketing Garden Mums, Ohio Florists' Association (1996)).

### SUMMARY OF THE INVENTION

The present invention relates to a new and distinct cultivar of chrysanthemum, Dendranthema x hybrida, referred to as 95-105-6. The present invention also relates to seed, pollen, ovules and cuttings from said chrysanthemum plant. Moreover, the present invention relates to a tissue culture comprising regenerable cells of chrysanthemum plant 95-105-6.

The present invention also relates to seed of a chrysanthemum plant 95-105-6 and to chrysanthemum plants produced by growing said seed.

The present invention also relates to a method of producing a first generation (F₁) hybrid chrysanthemum seed. The method involves crossing a first parent chrysanthemum plant with a second parent chrysanthemum plant and harvesting the resultant a first generation (F₁) hybrid chrysanthemum seed. Either or both of the first or second chrysanthemum plants may be chrysanthemum plant 95-105-6. Additionally, the present invention also relates to a first generation (F₁) hybrid chrysanthemum plant produced by growing the hybrid seed produced by said method.

The present invention also relates to a chrysanthemum plant having a lineage which includes chrysanthemum plant 95-105-6 and which exhibits at least one of the following characteristics: a shrub-like appearance and a massive floral display during its second year of growth, winter hardiness in zones 3-10, frost tolerant flower petals in zones 3-10 and/or self-pinching.

Finally, the present invention relates to viable chrysanthemum seeds and plants and succeeding generations thereof which are grown from seeds deposited under ATCC Accession number PTA-1115.

### BRIEF DESCRIPTION OF THE FIGURES

The file of this patent contains at least one drawing executed in color. Copies of this patent with color drawings(s) will be provided by the Patent and Trademark Office upon request and payment of the necessary fee.

FIG. 1 shows an adaxial and abaxial views of the leaf shape of chrysanthemum variety 95-105-6.

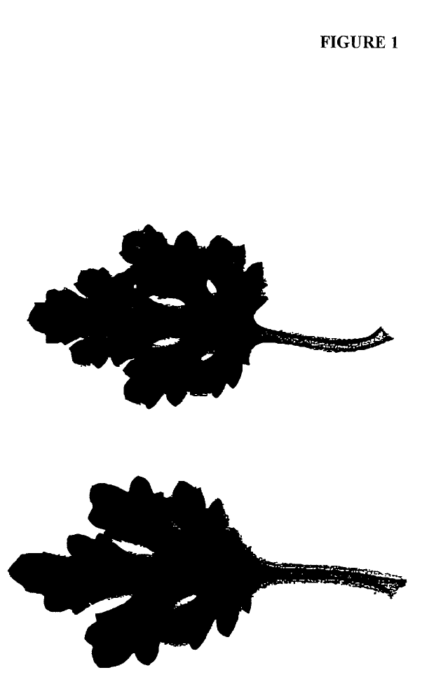

FIG. 2 shows the breeding history of chrysanthemum variety 95-105-6.

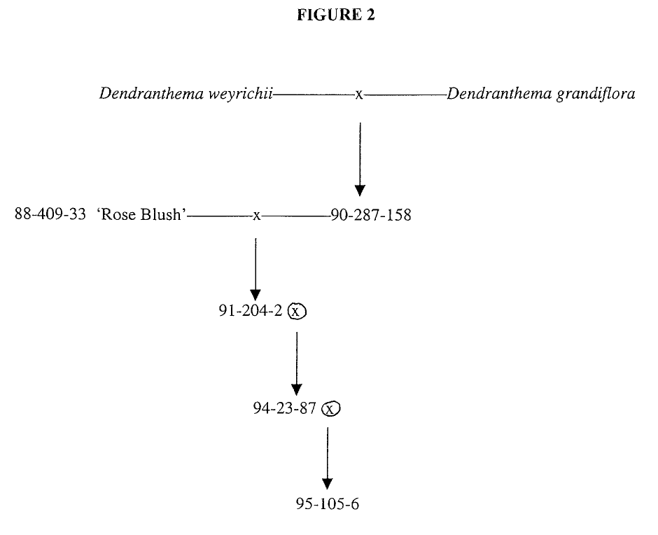

FIG. 3 is a color photograph of chrysanthemum variety 95-105-6 after one year of growth.

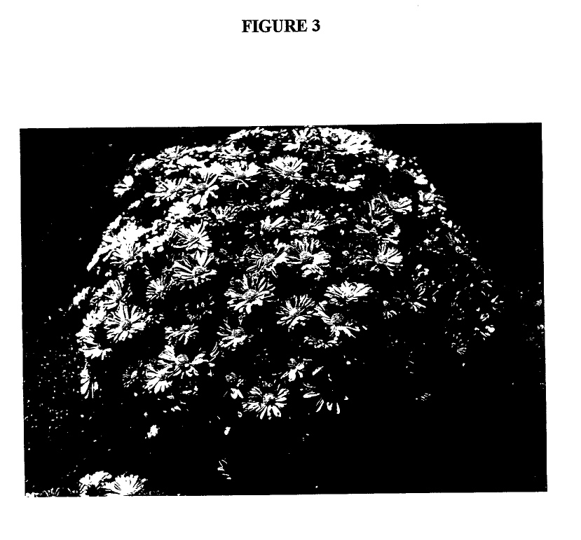

FIG. 4 is a color photograph of chrysanthemum variety 95-105-6 after two years of growth.

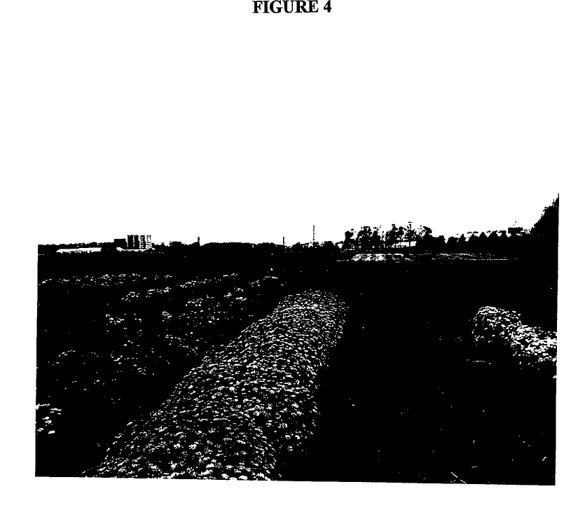

### DETAILED DESCRIPTION OF THE INVENTION

The present invention relates to a new and distinct cultivar of chrysanthemum, Dendranthema x hybrida, which is referred to herein as 95-105-6.

95-105-6 arose from breeding and research efforts in St. Paul, Minn. In 1989, a cross was made between Dendranthema weyrichii, which is commercially available from White Flower Farms, Connecticut, as the female parent, with Dendranthema grandiflora, which is commercially available from Yoder Brothers, Inc. Barberton, Ohio as the male parent and the resulting seeds, identified as 90-287-158, were collected. In 1990, a plant of 90-287-158 was crossed as the male parent with plants identified as 88-409-33, a University of Minnesota variety ‘Rose Blush’, as the female parent and the resulting seeds, identified as 91-204-2, were collected. In 1993, plants of 91-204-2 were selfed and the resulting seeds, identified as 94-23-87 were collected. In 1994, plants of 94-23-87 were selfed and the resulting seeds, identified as cross number 95-105 were collected. In 1995, seedlings of cross 95-105 were germinated and flowering progeny evaluated. 95-105-6 was the sixth plant from the cross and was selected during the fall of 1995.

95-105-6 possesses a number of unique characteristics. Specifically, 95-105-6 exhibits extreme hybrid vigor. In its first year after planting, when grown in the fall under natural daylength without the application of growth regulators, 95-105-6 develops into a plant having a plant height of about 14 to about 19 inches and a spread to about 2.0 feet. However, in the second and subsequent years after planting when grown in the fall under natural daylength without the application of growth regulators, 95-105-6 develops into a flowering herbaceous shrub having a plant height of from about 1.9 to about 2.4 feet and a spread from about 2.5 to about 6.0 feet. Moreover, during the fall season of the second year (August-October), 95-105-6 exhibits a massive floral display. Typically, each plant contains about 6,000 flowers. Additionally, the flowers of these plants, unlike other D. grandiflora hybrids, are slightly toned with grey, giving the flower petals a slightly altered coloration.

In addition to its shrub-like appearance and massive floral display, 95-105-6 exhibits superior winter hardiness when compared with other chrysanthemum varieties known in the art. Specifically, 95-105-6 has been shown to be hardy in zones 3-10 in uncovered field conditions without the need for added protection such as snow fences, mulch, etc. Additionally, the flower petals 95-105-6 exhibit frost tolerance, which extends the blooming season to the first hard freeze in the north (In zones 3-4, the first frost usually takes place between September 1-15. In zones 34, the first freeze usually takes place between October 1-20).

Another unique characteristic of 95-105-6 is that it is self-pinching. The fact that 95-105-6 is self-pinching is advantageous as it eliminates the need for labor intensive pinching practices employed early in the growing season in other chrysanthemum varieties in order to improve the branching of these plants.

95-105-6 is genetically stable and can be stably reproduced by means of asexual propagation. Samples of 95-105-6 thus far tested have been found to be stable through asexual propagation. Cuttings for asexual propagation can be taken at any time of the year and no special hormones or soil mixtures are required.

95-105-6 maintains functional male and female organs, and can thus be employed as a female and/or male parent in traditional plant breeding. Specifically, 95-105-6 can be used in crosses with other chrysanthemum varieties possessing commercially desirable phenotypes or as both the female and male parent to produce new chrysanthemum varieties. Therefore, the present invention contemplates a chrysanthemum plant having a lineage which includes chrysanthemum plant 95-105-6 and which exhibits one or more of the following characteristics: a shrub-like appearance and a massive floral display during its second year of growth, superior winter hardiness in zones 3-10, frost tolerant flower petals in zones 3-10 and self-pinching.

95-105-6 has not been observed under all possible environmental conditions. The phenotype may vary significantly with variations in environment such as temperature, light intensity and length, without, however, any variance in genotype.

The following examples are set forth as representations of specific and preferred embodiments of the present invention. These examples are not to be construed as limiting the scope of the invention in any manner. It should be understood that many variations and modifications can be made while remaining within the spirit and scope of the invention.

### EXAMPLE 1

### Description of the Female Parent: Dendranthema weyrichii—‘Pink Bomb’

Commercially available from: White Flower Farms, Connecticut

Plant Habit: Prostrate, spreads via rhizomes to form a large mat after the first year

Plant Dimensions: 1.5′ diameter, 5-6″ tall

USDA Hardiness: Zones 4-9 (Southeast)/Zone 10 (west)

Flower: Single daisy, light lavender-colored ray florets, central disc florets with yellow pollen

Leaves: Dark green, very shiny leaf surface (glossy), glabrous, leaf margins are deeply incised

Cultural Notes: Tends to rosette; needs cold treatment to flower consistently; flowering can be sporadic with gaps in the plant architecture; obligate short-day plant

### EXAMPLE 2

### Description of the Male Parent: Dendranthema grandiflora

Commercially available from: Yoder Brothers, Inc., Barberton, Ohio

Plant Habit: Cushion

Plant Dimensions: Similar to other cushion types commercially available from Yoder Brothers, Inc., such as, but not limited to the variety, ‘Soft Cherie’

USDA Hardiness: Zone 6-9 (Southeast)/Zone 10 (west)

Flower: Single or duplex daisy, possibly orange or bronze ray florets, central disc florets with yellow pollen

Leaves: Similar to other Yoder Brother, Inc. cushion series

Cultural Notes: Facultative short-day plant

### EXAMPLE 3

### Description of Pollination and Crossing Procedures for Dendranthema x hybrida

Chrysanthemums are members of the Asteraceae (Compositae) family, possessing flowers that are composite inflorescences of either ray (gynoecious, located around the periphery of each inflorescence) and/or disc (hermaphroditic, located in the center of the inflorescence) florets (National Chrysanthemum Society, 1957). Numerous flower forms are present within Dendranthema x hybrida, ranging from single daisy (containing one row of ray florets and multiple rows of disc florets) to fully double (containing 100% ray florets and 0% disc florets). The male and female parents described in Examples 1 and 2 and used in all pollinations performed were either single daisies; duplex (2 rows of ray florets), triple (3), quadriplex (4), pentaplex (5); or semi- (>5) to fully-double (>10). Floret number also varies widely depending on the flower form.

Both ray and disc florets are fertile with each containing a solitary ovule, capable of maturing into one seed/floret after fertilization and seed development. Ray and disc florets both contain one pistil with a 2-3 pronged stigma, a style, and an ovary. Ray florets, being gynoecious, are imperfect flowers since they lack stamens. The disk floret possesses five stamens that are united in a cylinder around the style. Pollen production occurs in two sacs on the inner surface of each anther.

A maturing single daisy flower form proceeds as follows. First, the outer concentric ring of florets (ray petals) become receptive. Pollinations on the female parent (described in Example 1) would be made using receptive stigmas on these ray petals at this stage. Second, on the next day the first outer ring of disc florets reach anthesis (anther dehiscence, pollen shed) and the pollen is pushed upward (for ease of pollen collection) by pistil elongation. Stigmas become receptive on these florets soon after emergence above the corolla tube, bearing the pollen aloft. Pollen would be collected from anthers on plants serving as male parents at this stage, as soon as pollen shed occurs. Third, pollen shed and stigmatic receptivity proceed sequentially within each concentric ring of disc florets, until terminating with the final ring of disc florets.

D. x hybrids possesses low levels of expression of self incompatibility, a genetically based physiological mechanism preventing fertilization of parents with matched S alleles. Thus, in some cases self pollinations were performed instead of outcross pollinations, following the procedures outlined below.

Laboratory Pollinations & Seed Ripening

Flowers from both the male and female parents from Examples 1 and 2 were chosen when the outer ring of ray florets were at a 45-90° angle, ensuring that no ray floret was receptive. Each flower was removed (cut) from the parental plant, leaving a 2-4° peduncle stem length. After the flowers were removed from each of the parental plants, the peduncle of each inflorescence was inserted into a styrofoam board floating on a solution of 1% sucrose and 200 ppm 8-hydroxy-quinoline citrate in the laboratory. These were located 20-30 cm below cool-white fluorescent lights (150 μmols s⁻¹m⁻²) with a 16-hour photoperiod (0600-2200 HR) at 29±1° C. temperature under laboratory conditions (Anderson, et al., 1990). Solutions were changed weekly. Inflorescences used for female and/or male parents in all pollinations were isolated from each other in the laboratory. No insects were present, as possible pollen vectors, in the laboratory. Use of these laboratory crossing conditions has been demonstrated to reduce or eliminate source-sink interactions (that can impact seed production) and maximize seed maturation times (Anderson, et al., J. Amer. Soc. Hort. Sci. 115(2):329-336 (1990)).

Cross-Pollinations

Emasculation protocols for each female parent was as follows. All ray floret petals were trimmed back just above the location of each stigma, using curved, fine scissors. After trimming, the stigma would continue development and emerge above the cut surface ≧1 day(s) later. For cross pollinations, all disc florets were cut out of the inflorescence just above the ovary to prevent any self pollen contamination. Inflorescences were tagged after emasculation to identify the female and male parents for each cross.

Upon reaching receptivity, pollen was applied to stigmatic surfaces of each receptive ray floret. Each inflorescence was pollinated each day, depending on the number of rings of ray florets present.

Pollen collection was accomplished using fine camel hair brushes if pollen shed was copious. In cases of minimal pollen production on a male parent, a vacuum-assisted pollen collector was used to maximize pollen collection.

Self-Pollinations

Each inflorescence used for self-pollinations (inbreeding) was treated with the following modified procedures. All ray floret petals were trimmed back just above the location of each stigma, using curved, fine scissors. After trimming, the stigma would continue development and emerge above the cut surface ≧1 day(s) later. Disc florets were not removed; they remained with the inflorescence. Inflorescences were tagged after emasculation to identify the female and male parents for each self pollination. For self pollinations, the same inflorescence served as both the male and female parent. Following pollen shed of the outer-most ring of disc florets, pollen was collected and applied daily to the receptive ray and disc floret stigmas.

Seed Development

Following fertilization, seed development occurred over a 4-6 week period. As soon as the base of the receptacle for each inflorescence became brown, the inflorescences were harvested and dried at 21° C. for 1-2 weeks. Individual inflorescences were lightly macerated (between the thumb and forefinger) to disconnect the mature seeds from the chaff (dried stigma/styles, corolla tubes, anthers, etc.). Seeds were then separated from the chaff by using an air column.

### EXAMPLE 4

### Greenhouse Observations of Chrysanthemum Variety 95-105-6

The following observations and measurements describe plants grown in controlled greenhouse areas in St. Paul, Minn. (45° N latitude) under natural temperature and daylength prevailing during May through June. Additionally, in the following description, color references are made to The Royal Horticultural Society Colour Chart. The color values were determined on plant material grown in St. Paul, Minn. on Oct. 15, 1999.

Propagation

Type Herbaceous stem cutting

Time to rooting About 1 week

Rooting habit Vigorous

Plant Description

Appearance, shape

Flattened mound (first year)

Spherical mound (second year)

Appearance, growth habit Mound

Appearance, growth rate/vigor Vigorous

Plant height

about 14 to about 19 inches (first year)

about 1.9 to about 2.4 feet (second year)

Lateral branch length 1 to 3 feet

Quantity of lateral branches after removal One per node of apical meristem

Stem color RHS Scheeles Green 860/3

Foliage Description

Number of leaves per plant Greater than 10,000 (second year)

Number of leaves per lateral branch 5 to 25

Leaf arrangement Alternate

Leaf size, fully expanded, length 9.7 cm

Leaf size, fully expanded, width 4.6 cm

Leaf apex Obtuse to mucronate

Leaf base Cuneate to oblique

Leaf margin Incised (Mulberry-like incisions)

Leaf texture Glabrous

Petiole length 3.8 cm

Color, young foliage adaxial surface RHS Scheeles Green 860

Color, young foliage abaxial surface RHS Spinach Green o960/3

Color, fully expanded foliage adaxial surface RHS Ivy Green ooo1060/3

Color, fully expanded foliage abaxial surface RHS Parsley Green oo962/2

Color, venation adaxial surface RHS Lavender Green ooo761/1

Color, venation abaxial surface RHS Lavender Green ooo761/2

Color, petiole RHS Spinach Green o960

Inflorescence Description

Appearance Head (composite), triplex-quadriplex daisy

Flowering response About 5.5 weeks (SD)

Quantity of inflorescences

About 800 (first year)

About 6,000 (second year)

Inflorescence size, diameter 6.3 cm

Inflorescence size, depth (height) 2.3 cm

Inflorescence size, diameter of disc 1.5 cm

Opening inflorescences, bud shape Flattened hemisphere to widened upright tubular

Opening inflorescences, bud size, length 0.7 cm

Opening inflorescences, bud size, width 0.9 cm

Opening inflorescences, bud color RHS Pansy Purple 928/1

Ray florets, shape Linear lanceolate

Ray florets, size, length 3 cm

Ray florets, size, width 0.7 cm

Ray florets, apex Multi-notched mucronulate

Ray florets, base Attenuate

Ray florets, margin Entire

Ray florets, texture Glabrous

Ray florets, aspect From about 45° vertical to slightly pendant 45°

Number of ray florets per inflorescence About 60

Ray florets, color, when opening, adaxial surface RHS Garnet Lake 828

Ray florets, color, when opening, abaxial surface RHS Chrysanthemum Crimson 824

Ray florets, color, mature, adaxial surface RHS Indian Lake 826/2

Ray florets, color, mature, abaxial surface RHS Erythrite Red oo27/2

Ray florets, color, fading to RHS Erythrite Red oo27/3

Disc florets, shape Tubular, rounded at tip

Disc florets, size, length 0.6 cm

Disc florets, size, width 0.4 cm

Number of disc florets per inflorescence About 191

Disc florets, color, immature RHS Straw Yellow 604

Disc florets, color, mature RHS Maize Yellow 607/1 with an eye in the center of each disc which is RHS Oxblood Red oo823

Peduncle, aspect, strength Stiff

Peduncle, aspect, angle to stem 30°

Peduncle, length, first peduncle 4.2 cm

Peduncle, length, fourth peduncle 7 cm

Peduncle, texture Slightly hirsute

Peduncle, color RHS Spinach Green o960/2

Reproductive organs, androecium, floret location Disc florets

Anther color RHS Chinese Yellow 606/1

Pollen, abundance Abundant

Pollen, color RHS Buttercup Yellow 5

Reproductive organs, gynoecium, floret location Disc/ray florets

Style color RHS Egyptian Buff 407/1

Disease Resistance None Known

Seed Production About 251 ovules/flower

Deposit Information

Two thousand five hundred (2500) seeds of chrysanthemum variety 95-105-6 have been placed on deposit with the American Type Culture Collection (ATCC), 10801 University Blvd., Manassas, Va., 20110-2209 under Deposit Accession Number PTA-1115 on Dec. 30, 1999. This deposit was made in compliance with the Budapest Treaty requirements that the duration of the deposit should be for thirty (30) years from the date of deposit or for five (5) years after the last request for the deposit at the depository or for the enforceable life of a U.S. Patent that matures from this application, whichever is longer. These chrysanthemum seeds will be replenished should it become non-viable at the depository.

The present invention is illustrated by way of the foregoing description and examples. The foregoing description is intended as a non-limiting illustration, since many variations will become apparent to those skilled in the art in view thereof It is intended that all such variations within the scope and spirit of the appended claims be embraced thereby.

Changes can be made to the composition, operation and arrangement of the method of the present invention described herein without departing from the concept and scope of the invention as defined in the following claims.

What is claimed is:

1. A new and distinct Chrysanthemum plant designated 95-105-6.

2. Seed of the plant of claim 1.

3. Pollen of the plant of claim 1.

4. An ovule of the plant of claim 1.

5. A tissue culture comprising regenerable cells of the plant of claim 1.

6. A cutting of the plant of claim 1.

7. Seed of a chrysanthemum plant designated 95-105-6.

8. A chrysanthemum plant produced by growing the seed of claim 7.

9. A method for producing F₁ hybrid chrysanthemum seed, the method comprising the steps of crossing a first parent chrysanthemum plant with a second parent chrysanthemum plant and harvesting the resultant F₁ hybrid chrysanthemum seed, wherein either the first or second chrysanthemum plant is the chrysanthemum plant of claim 1.

10. A first generation hybrid chrysanthemum plant produced by growing the hybrid chrysanthemum seed of claim 9.

11. A method for producing a first generation hybrid chrysanthemum seed, the method comprising the steps of crossing a first parent chrysanthemum plant with a second parent chrysanthemum plant and harvesting the resultant first generation hybrid chrysanthemum seed, wherein the first and second chrysanthemum plant is the chrysanthemum plant of claim 1.

12. A first generation hybrid chrysanthemum plant produced by growing the hybrid chrysanthemum seed of claim 11.

13. A chrysanthemum plant having a lineage which includes chrysanthemum plant 95-105-6 and which exhibits at least one of the following characteristics selected from the group consisting of: a shrub-like appearance and a massive floral display during its second year of growth, winter hardiness in zones 3-10, frost tolerant flowers in zones 3-10 and self-pinching.

14. Viable chrysanthemum seeds and plants and succeeding generations thereof grown from seeds deposited under ATCC Accession number PTA-1115.
